(19) United States
(12) Patent Application Publication
(10) Pub. No.: US 2026/0320441 A1
(43) Pub. Date: Oct. 15, 2026

(54) SYSTEM AND METHOD FOR GENERATING NON-THERAPEUTIC COMPARATIVE ENGAGEMENT OUTPUTS FROM MULTIMODAL PHYSIOLOGICAL SIGNAL PROCESSING AND INFERENCE-BASED ENGAGEMENT VECTOR COMPUTATION CROSS-REFERENCE TO RELATED APPLICATIONS

(71) Applicants: Hyun Jung Lee, Carmel, IN (US); Sung Hoon Lee, Carmel, IN (US); Yoo Kyung Lee, Carmel, IN (US)

(72) Inventors: Hyun Jung Lee, Carmel, IN (US); Sung Hoon Lee, Carmel, IN (US); Yoo Kyung Lee, Carmel, IN (US)

(21) Appl. No.: 19/645,140

(22) Filed: Apr. 11, 2026

Related U.S. Application Data
(60) Provisional application No. 63/787,574, filed on Apr. 11, 2025.

(51) Int. Cl. G06N 5/04; A61B 5/00; A61B 5/16; G01D 9/00

(52) U.S. Cl. CPC G06N 5/04; A61B 5/165; A61B 5/7267; G01D 9/005; A61B 2503/12

(57) ABSTRACT

A computer-implemented system and method generate comparative non-therapeutic engagement outputs for evaluation of candidate stimuli. Time-series physiological signals are acquired from one or more wearable sensing devices during user interaction with physical, digital, or multisensory stimuli. The signals are preprocessed using modality-specific operations and used to generate a synchronized multimodal feature representation derived from at least two physiological signal modalities. A trained computational inference model performs non-linear multimodal fusion to generate a continuous multi-dimensional engagement vector representing a quantified physiological interaction response of the individual user. One or more similarity or distance measures are computed between the engagement vector and reference engagement vectors associated with the same individual user. A structured comparative output is generated encoding relative distances in engagement-vector space as a non-ranked relational representation to support user-driven comparison without prescribing a selection.

### CROSS-REFERENCE TO RELATED APPLICATIONS

This application claims priority to U.S. Provisional Patent Application 63/787,574 filed Apr. 11, 2025, entitled “System and Method for Emotion-Based Engagement and Decision Support Using Wearable Biofeedback in Multi-Sensory Product Environments,” the entire contents of which are incorporated herein by reference.

### STATEMENT REGARDING FEDERALLY SPONSORED RESEARCH

Not applicable.

### FIELD OF THE INVENTION

The present invention relates generally to computer-implemented systems and methods for processing multimodal physiological signals captured from wearable sensing devices during a user's interaction with physical, digital, or multisensory stimuli. More particularly, the invention relates to computing continuous multi-dimensional engagement vectors from multimodal physiological signals using non-linear computational inference models, comparing those vectors against stored reference engagement vectors associated with the same individual user, and generating structured comparative outputs that encode relative distances in engagement-vector space for non-therapeutic decision-support applications. In certain embodiments, the system may operate using a within-user reference architecture in which engagement-vector representations are generated from the individual user's own physiological interaction history; a same-session initialization mechanism enabling first-use operation without cross-user bootstrapping; a trained computational inference model whose parameters remain fixed during runtime deployment; a post-inference personalization layer operating on inference-model-generated vectors without modifying model parameters; and a structured comparative output encoding relative engagement distances in engagement-vector space to support user-driven evaluation and purchase decision-making with respect to physical, digital, or multisensory stimuli—including commercial aesthetic objects such as artworks, fragrances, furniture, apparel, and related categories. The system is not directed to emotional state classification, clinical diagnosis, or therapeutic intervention.

### BACKGROUND OF THE INVENTION

Human responses to aesthetic and sensory stimuli—including artworks, design objects, materials, fragrances, soundscapes, lighting conditions, and spatial environments—are complex, multimodal, and dynamic. Existing approaches to understanding such responses rely primarily on subjective self-reporting, post-experience surveys, or indirect behavioral proxies, which are often biased, inconsistent, or poorly correlated with real-time physiological response.

Certain known systems attempt to infer affective or emotional states using physiological signals. These systems, however, commonly exhibit one or more technical limitations that distinguish them from the present invention.

First, prior systems directed at physiological engagement measurement frequently rely on a single physiological signal modality—such as electroencephalography (EEG) alone, electrodermal activity (EDA) alone, or event-related potential (ERP) alone—without performing non-linear fusion across two or more distinct modalities. Single-modality systems produce outputs that are architecturally distinct from the multimodal engagement vectors generated by the present invention.

Second, prior systems commonly generate outputs in the form of discrete emotional state classifications, affective category labels, population-level stimulus efficacy predictions, or system-generated product recommendations. These output forms differ fundamentally from the structured comparative outputs of the present invention, which encode relative distances in a multi-dimensional engagement-vector space. In certain embodiments, the structured comparative outputs are configured to avoid generation of ordered or ranked lists of candidate stimuli, recommendation scores, or population-level predictive models.

Third, prior systems that produce comparative or evaluative outputs with respect to stimuli commonly derive those outputs from physiological responses of multiple subjects —constructing a population-level or group-level stimulus efficacy model and predicting stimulus effectiveness for a given subject relative to that population model. Such systems are architecturally distinguished from the present invention, which generates structured comparative outputs based exclusively on engagement vectors derived from the individual user's own physiological responses, compared against reference engagement vectors generated for the same individual user during prior or current interaction sessions.

Fourth, prior recommendation and decision-support systems based on physiological data commonly employ direct single-modality biometric mapping to produce product recommendations, without performing non-linear multimodal fusion through a trained computational inference model. In certain embodiments, systems employing: (a) non-linear multimodal fusion of at least two physiological signal modalities through a trained computational inference model; (b) generation of a continuous multi-dimensional engagement vector from the individual user's own physiological response; (c) comparison of that vector against reference engagement vectors derived from the same individual user's prior or current-session interaction history; (d) a post-inference personalization layer operating exclusively on inference-model-generated vectors without modifying inference model parameters at runtime; and (e) structured comparative output encoding relative engagement distances and, in certain embodiments, without generating ranked or ordered lists—may provide individual-specific physiological engagement outputs for non-therapeutic decision support in the evaluation and selection of commercial aesthetic objects including artworks, fragrances, musical compositions, furniture, spatial design environments, apparel, footwear, headwear, jewelry, and accessories.

Accordingly, there exists a need for a non-therapeutic, computer-implemented system capable of transforming multimodal physiological signals into continuous multi-dimensional engagement vectors through non-linear inference, comparing those vectors against within-user reference engagement vectors, and generating structured comparative outputs that encode relative engagement distances for individual user decision support without generating ranked lists, emotional classifications, or population-level efficacy predictions.

### BRIEF SUMMARY OF THE INVENTION

In certain embodiments, the system may include: (1) a within-user reference vector architecture in which reference engagement vectors are generated from the individual user's own physiological interaction history, with no cross-user data incorporated into individual user outputs; (2) a same-session initialization mechanism enabling first-use operation by generating initialization reference vectors from the current session's physiological signals, without cross-user bootstrapping; (3) a fixed trained computational inference model whose parameters are not modified during runtime deployment, with user-specific adaptation performed exclusively by a downstream personalization layer operating on inference-model-generated vectors; (4) non-linear multimodal fusion of physiological signals from at least two distinct modalities through a trained computational inference model, generating a continuous multi-dimensional engagement vector; and (5) a structured comparative output encoding relative distances in engagement-vector space without generating an ordered or ranked list of candidate stimuli. In certain embodiments, the system supports user-driven evaluation and purchase decision-making in aesthetic and experiential product domains including art, design, fragrance, fashion, and related categories. In certain embodiments, the structured comparative output is presented in a format that enables direct comparison of candidate stimuli during an interaction session without prescribing a selection or ranking.

The present invention provides a computer-implemented system and method for generating comparative non-therapeutic engagement outputs associated with an individual user's interaction with physical, digital, or multisensory stimuli. The system captures time-series physiological signals from one or more wearable sensing devices, processes those signals through a modality-independent preprocessing pipeline, and applies a trained computational inference model performing non-linear multimodal fusion to generate a continuous multi-dimensional engagement vector representing a quantified physiological interaction response of the individual user.

The generated engagement vector does not constitute a classification of a discrete emotional or affective state, is not generated by comparison to stored baseline physiological responses associated with predefined emotional categories, and is not derived solely from neural similarity measures across subjects.

The system computes one or more similarity or distance measures between the continuous multi-dimensional engagement vector and one or more reference engagement vectors associated with the individual user. The reference engagement vectors comprise vectors generated during prior interaction sessions of the same individual user with candidate stimuli, or, in the absence of prior stored reference engagement vectors for the same individual user, initialization reference vectors generated from physiological signals acquired during the current interaction session. Such initialization reference vectors are generated exclusively from physiological signals of the same individual user and do not incorporate cross-user data.

Based on the computed similarity or distance measures, the system generates a structured comparative output for the candidate stimuli. The structured comparative output comprises data encoding relative distances in a multi-dimensional engagement-vector space between one or more candidate stimuli and the same individual user's reference engagement vectors. In certain embodiments, the structured comparative output is configured to avoid generation of an ordered or ranked list of candidate stimuli. The structured comparative output is generated such that the output generation pipeline does not apply, at any stage from engagement-vector comparison through structured comparative output generation, any model, parameter, or transformation derived from cross-user engagement data or trained on or derived from physiological responses, engagement data, or preference data from a plurality of users other than the individual user that directly or indirectly determines, contributes to, or modifies the structured comparative output for the individual user, wherein this limitation does not preclude the use of multi-user data during model training or for aggregate analytics that do not generate individual user structured comparative outputs.

The output provides non-therapeutic comparative decision support and does not constitute a population-level predictive output or a clinical, diagnostic, or therapeutic output. In certain embodiments, the system further generates a graphical mapping of engagement vectors in a continuous multi-dimensional space, representing relative engagement distances between candidate stimuli and the same individual user's reference engagement vectors, without assigning ranked positions or priority scores. In certain embodiments, the system further applies a personalization module that operates exclusively on historical engagement vectors output by the inference engine for the individual user, without modifying inference model parameters during runtime. In certain embodiments, the system aggregates continuous multi-dimensional engagement vectors for the same individual user across multiple interaction sessions to generate longitudinal engagement metrics for non-therapeutic comparative analysis.

The disclosed system is expressly non-therapeutic. It does not diagnose, treat, mitigate, prevent, or monitor any medical, psychological, or psychiatric condition and does not generate therapeutic, clinical, or diagnostic recommendations.

### BRIEF DESCRIPTION OF THE DRAWINGS

FIG. 1 is a block diagram illustrating a system architecture for acquiring and processing physiological signals, according to one embodiment.

FIG. 2 is a flowchart illustrating a data-processing pipeline for generating engagement vectors from physiological data.

FIG. 3 illustrates representative time-series physiological signals acquired by a wearable sensing device.

FIG. 4 is a block diagram illustrating a processor-implemented inference engine architecture for generating engagement vectors.

FIG. 5 illustrates example user-interface views representing structured comparative decision-support outputs.

FIG. 6 is a block diagram illustrating a personalization module configured to apply post-inference operations to user-specific engagement vectors.

FIG. 7 is a flowchart illustrating a decision-support and comparative output workflow.

FIG. 8 is a block diagram illustrating a cloud-based remote system architecture for distributed processing of engagement data.

FIG. 9 is a flowchart illustrating a group-level aggregation workflow for generating aggregate designation outputs.

FIG. 10 is a block diagram illustrating a hybrid processing configuration for distributed physiological signal acquisition and inference execution.

### DETAILED DESCRIPTION OF THE INVENTION

### A. System Overview

The disclosed system is a computer-implemented system configured to generate comparative non-therapeutic engagement outputs associated with an individual user's interaction with a physical, digital, or multisensory stimulus. The system comprises four principal functional components: (a) one or more wearable sensing devices; (b) at least one processor configured for preprocessing and multimodal feature extraction; (c) an inference engine implementing a trained computational inference model; and (d) a computer interface providing a decision-support module.

The wearable sensing devices are configured to generate time-series physiological signals from the individual user during interaction with the stimulus. Physiological signals may be acquired across at least two physiological signal modalities selected from electrodermal activity, heart rate, heart-rate variability, skin temperature, photoplethysmography, respiration, electroencephalography, or motion data.

The at least one processor applies modality-specific signal preprocessing operations independently for each physiological signal modality, extracts a synchronized multimodal feature representation from the preprocessed signal segments, and provides the feature representation as input to the inference engine.

The inference engine executes a trained computational inference model performing non-linear multimodal fusion on the multimodal feature representation to generate a continuous multi-dimensional engagement vector representing a quantified physiological interaction response of the same individual user to the stimulus.

The computer interface outputs the continuous multi-dimensional engagement vector to a decision-support module configured to compute one or more similarity or distance measures between that vector and stored reference engagement vectors associated with the same individual user, and to generate a structured comparative output for candidate stimuli based on the computed measures. The structured comparative output encodes relative distances in engagement-vector space and, in certain embodiments, is configured such that it does not generate an ordered or ranked list of candidate stimuli and instead generates relational comparative representations encoding relative engagement distances in engagement-vector space, to support user-driven evaluation and decision-making with respect to candidate stimuli including commercial aesthetic objects.

The accompanying drawings illustrate exemplary embodiments and are not intended to limit the scope of the invention.

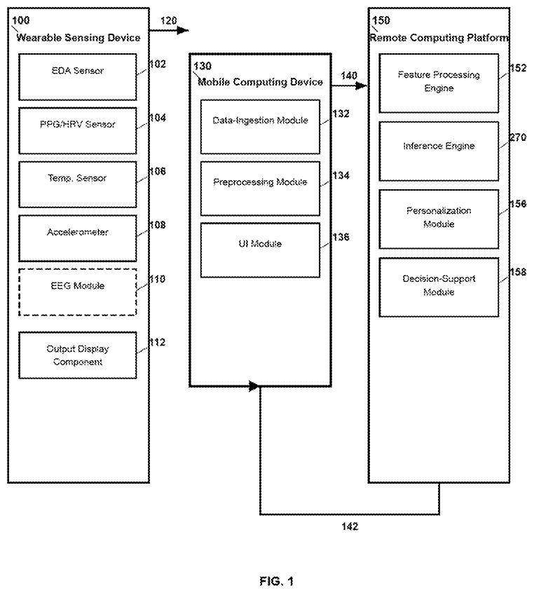

FIG. 1 illustrates an example system architecture comprising a wearable sensing device 100, a mobile computing device 130, and a remote computing platform 150.

The wearable sensing device 100 includes one or more physiological sensors, such as an electrodermal activity (EDA) sensor 102, a photoplethysmography (PPG) and heart-rate-variability (HRV) sensor 104, a temperature sensor 106, an accelerometer 108, and optionally an electroencephalography (EEG) module 110. The wearable sensing device 100 is configured to acquire, buffer, or transmit physiological signal data. In a preferred embodiment, the wearable sensing device 100 does not execute a trained computational inference model. The wearable sensing device 100 may further include an output display component 112 configured to receive and render structured comparative outputs; such rendering does not constitute execution of a trained computational inference model.

Physiological signals 120 acquired by the wearable sensing device 100 are transmitted to the mobile computing device 130. The mobile computing device 130 may include various functional modules, such as data-ingestion, preprocessing, or user-interface components. In certain embodiments, the mobile computing device 130 performs initial preprocessing and feature-extraction operations.

Processed physiological data or feature representations 140 are transmitted from the mobile computing device 130 to the remote computing platform 150. The remote computing platform 150 includes a feature-processing engine 152, a computational inference engine 270 configured to generate engagement-related response variables (engagement vectors), an optional post-inference personalization module 156, and a decision-support module 158.

Non-therapeutic decision-support outputs or engagement vectors 142 are returned from the remote computing platform 150 to the mobile computing device 130 for presentation. In certain embodiments, structured comparative outputs are additionally transmitted to and rendered by the wearable sensing device 100 via the output display component 112.

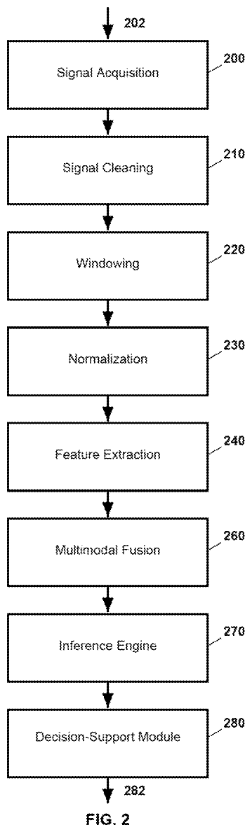

FIG. 2 illustrates a data-processing pipeline. The process begins with physiological signal acquisition 200. The acquired signals undergo signal-cleaning operations 210, which may include filtering, wavelet denoising, motion-artifact compensation, or drift correction. The cleaned signals are grouped into time windows 220 and subjected to normalization 230. This normalization may involve z-scoring, min-max scaling, detrending, or adaptive baselining applied independently to each physiological signal modality.

Following normalization, a feature extraction stage 240 computes specific physiological features, such as electrodermal, heart-rate-variability, temperature, accelerometer, or electroencephalography features. A multimodal fusion stage 260 then combines these features using methods such as weighted averaging, neural-attention mechanisms, or nonlinear transformations.

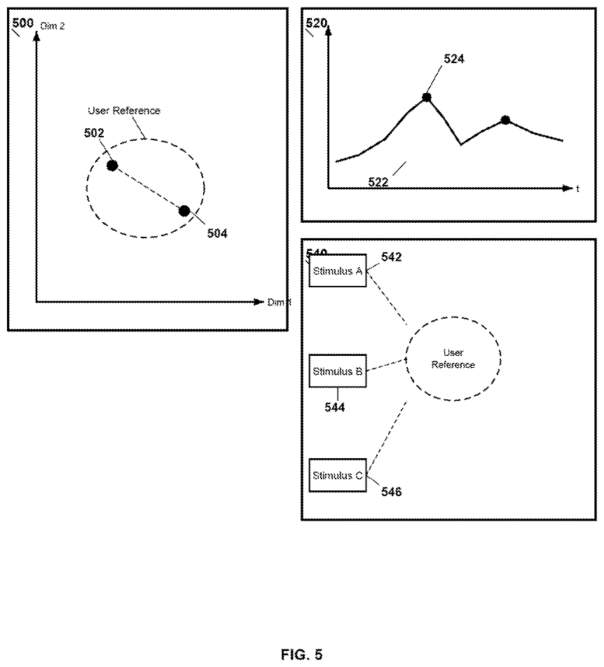

The resulting fused representation is provided to an inference engine 270 to compute engagement vectors. These engagement vectors are then provided to a decision-support module 280 to generate structured comparative outputs, which may encode relative engagement distances in an engagement-vector space, such as those illustrated in FIG. 5.

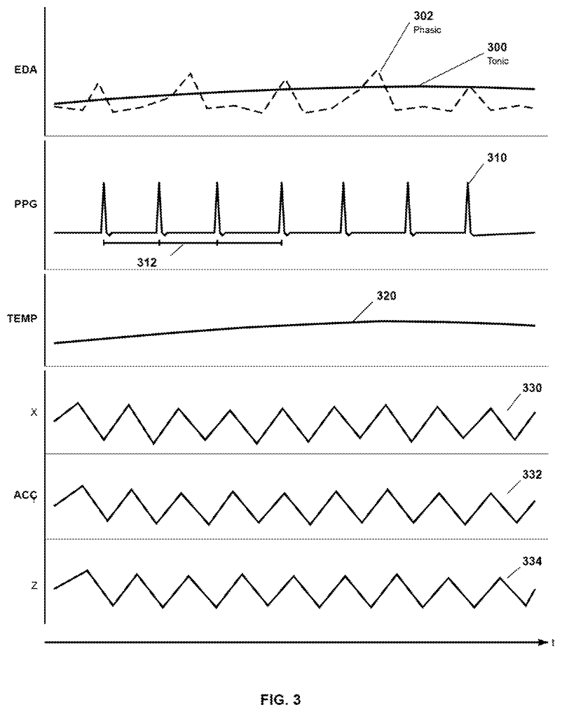

FIG. 3 illustrates representative time-series physiological signals obtained from the wearable sensing device 100. Electrodermal activity (EDA) signals may include a tonic component 300 and a phasic component 302. A photoplethysmography (PPG) waveform 310 and corresponding beat-to-beat heart-rate-variability intervals 312 are also shown. A temperature signal 320 and accelerometer (ACC) channels X 330, Y 332, and Z 334 are illustrated. Each channel is plotted with a horizontal time axis and a vertical amplitude axis. These signals represent the raw or minimally processed data that feed into the pipeline of FIG. 2.

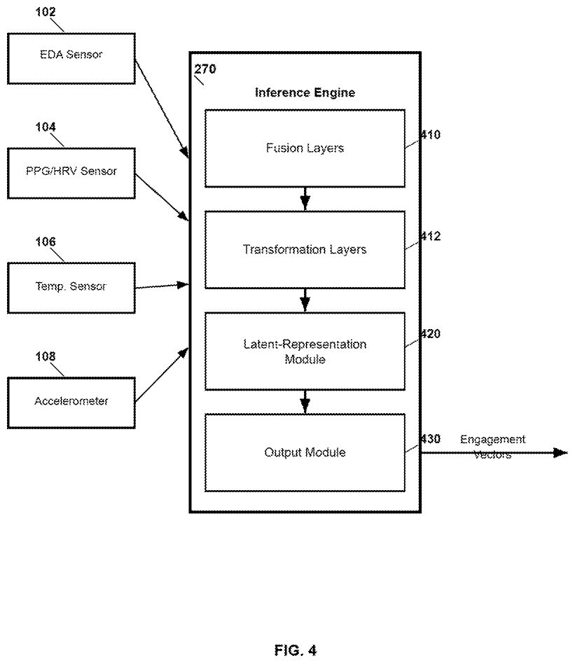

FIG. 4 illustrates a processor-implemented inference engine 270 configured to receive multimodal physiological feature representations and generate engagement vectors. The inference engine 270 includes one or more fusion layers 410 that integrate multimodal physiological features, such as EDA, PPG, and temperature features. One or more transformation layers 412 perform nonlinear processing to map features into a latent space. A latent-representation module 420 encodes intermediate interaction-response characteristics.

The inference engine 270 outputs engagement vectors through an output module 430. These engagement vectors are structured for downstream similarity or distance computation in an engagement-vector space and, in the illustrated embodiment, do not constitute a classification of a discrete emotional or affective state. Optional affect-related descriptors may be generated in downstream analysis layers outside the inference engine 270 for visualization purposes, but do not form part of the primary engagement vector.

FIG. 5 illustrates example user-interface views representing non-therapeutic structured comparative decision-support outputs. A two-dimensional engagement visualization 500 positions one or more candidate-stimulus response points 502 and 504 within a multi-dimensional engagement-vector space based on computed engagement vectors. The spatial positioning of response points encodes relative engagement distances between candidate stimuli and the individual user's reference engagement vectors in the engagement-vector space. In the illustrated embodiment, this is performed without assigning ranked positions or priority scores.

A time-series engagement graph 520 displays an engagement metric line 522, with peaks 524 indicating relative changes in user interaction intensity over time during interaction with a stimulus.

A comparative engagement distance interface 540 displays one or more candidate stimuli 542, 544, and 546 positioned relative to one another and to the individual user's reference engagement vectors based on computed similarity or distance measures. The interface presents structured comparative outputs encoding relative engagement distances and is configured to support a user's interpretation and decision-making. The system is configured to present these outputs without assigning rank, priority, or recommendation scores, and without prescribing a selection or ordered list of candidate stimuli.

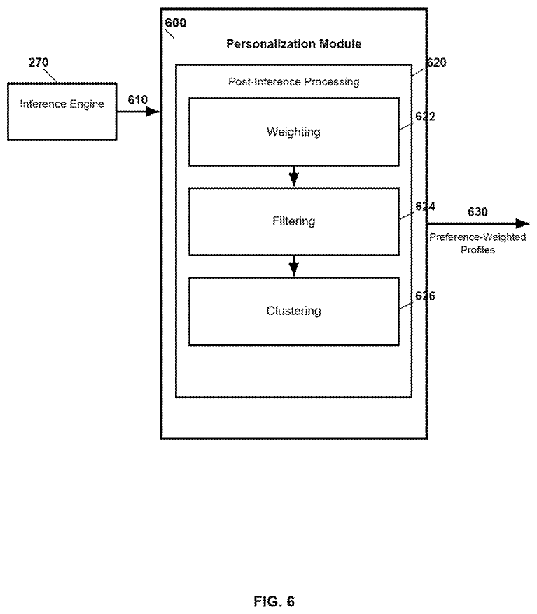

FIG. 6 illustrates a personalization module 600 configured to apply post-inference operations to historical engagement vectors associated with an individual user. The personalization module 600 receives engagement vectors 610 output by the inference engine 270 (shown in FIG. 1 and FIG. 4) from prior interaction sessions.

Post-inference processing 620 applied by the personalization module 600 may include weighting 622, filtering 624, or clustering 626 operations. These operations are applied exclusively to stored engagement vectors associated with the individual user, without incorporating data from other users. The personalization module 600 outputs adjusted engagement-vector-based representations or preference-weighted engagement profiles 630 for use in generating structured comparative outputs.

In a preferred embodiment, the personalization module 600 operates exclusively on stored engagement vectors output by the trained computational inference model for the individual user and does not modify parameters of the trained computational inference model during runtime. Consequently, inference model parameters remain fixed across users and sessions. In certain embodiments, clustering operations 626 are performed exclusively on a single user's across-session data to characterize intra-user engagement patterns without incorporating engagement vectors from other users into the clustering computation.

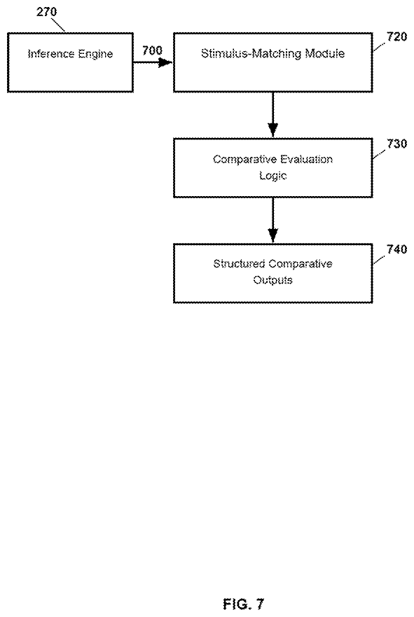

FIG. 7 illustrates a decision-support and comparative output workflow. Engagement vectors 700 generated by the inference engine 270 are provided to a stimulus-matching module 720. The stimulus-matching module 720 compares these engagement vectors against stored reference engagement vectors associated with the same individual user.

Comparative evaluation logic 730 operates independently of the inference engine 270 and applies one or more similarity or distance measures to generate non-therapeutic structured comparative outputs 740. These outputs encode relative engagement distances between candidate stimuli and the individual user's reference engagement vectors in an engagement-vector space.

In a preferred embodiment, the structured comparative outputs 740 do not constitute an ordered or ranked list of candidate stimuli. The decision-support workflow does not apply any model, parameter, or transformation derived from cross-user engagement data. All operations, from engagement-vector comparison through structured comparative output generation, are performed without relying on data from a plurality of users other than the individual user. Furthermore, the workflow does not rely on the classification of discrete emotional or affective states and remains functional in the absence of affect-related descriptors.

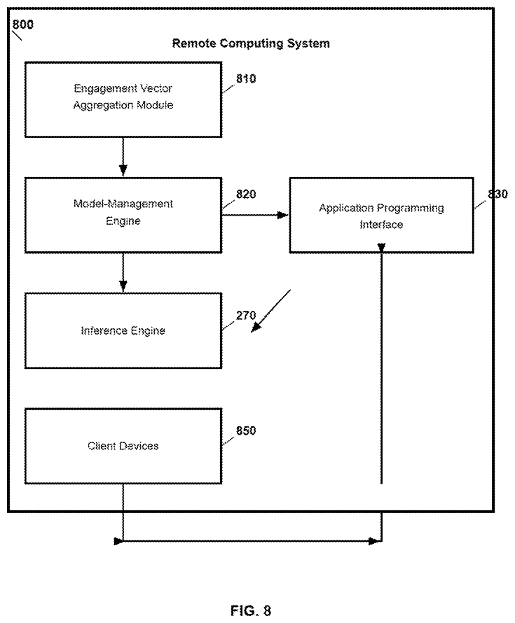

FIG. 8 illustrates a cloud-based and distributed implementation of a remote computing system 800. The remote computing system 800 includes an engagement vector aggregation module 810, a model-management engine 820, and an application-programming interface (API) 830. The API 830 facilitates communication with an inference engine 270 (previously described in FIGS. 2 and 4).

The model-management engine 820 is configured to manage inference-model versioning, selection, or deployment. In a preferred embodiment, this management is performed without user-specific retraining during runtime. Client devices 850 communicate with the remote computing system 800 via the API 830 to transmit feature representations and to receive engagement-related response variables or structured comparative outputs.

Personalization operations, as described in FIG. 6, are applied post-inference using stored engagement-related response variables associated with the individual user. In certain embodiments, the architecture of the remote computing system 800 is implemented as a centralized platform providing non-therapeutic analytics and decision-support services to a plurality of client devices 850.

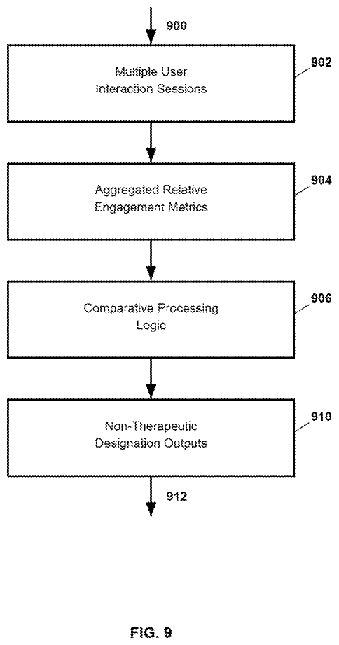

FIG. 9 illustrates a group-level aggregation workflow. Inference-model-generated engagement vectors across multiple user interaction sessions 902 are aggregated to generate aggregated relative engagement metrics 904. In a preferred embodiment, this aggregation is performed after the removal of individual-level identifying information to ensure anonymity.

The aggregated engagement metrics 904 are processed using comparative processing logic 906 to generate non-therapeutic designation outputs 910 associated with specific products, environments, or stimuli. These aggregated metrics are not used to generate individual user outputs and do not constitute a population-level stimulus efficacy model.

In certain embodiments, a parallel workflow may be implemented wherein hardware-configuration or device-capability metrics are evaluated using capability-assessment logic and compatibility mapping to generate non-therapeutic designation outputs associated with specific sensing configurations.

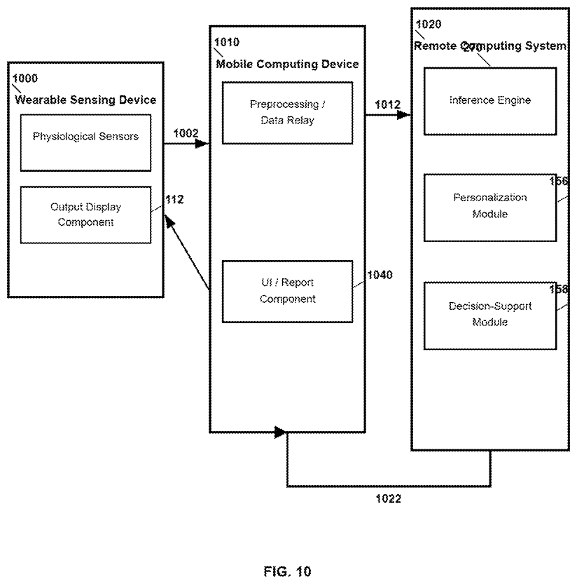

FIG. 10 illustrates a hybrid processing configuration. Physiological signal acquisition is performed on a wearable sensing device 1000, and preprocessing or data relay is performed on a mobile computing device 1010. Execution of the trained computational inference model is performed exclusively on a remote computing system 1020.

The wearable sensing device 1000 is configured to acquire, buffer, or transmit physiological signal data. In a preferred embodiment, the wearable sensing device 1000 does not execute a trained computational inference model. In certain embodiments, the wearable sensing device 1000 is further configured to receive and render structured comparative outputs generated by the remote computing system 1020 via transmission through the mobile computing device 1010 or directly. Such rendering of outputs does not constitute execution of the trained computational inference model.

Engagement vectors generated by the remote computing system 1020 are transmitted to the mobile device 1010 for presentation via a non-therapeutic user-interface or report component 1040. In certain embodiments, structured comparative outputs are additionally transmitted to and rendered by the wearable sensing device 1000.

### B. Non-Therapeutic Scope

The system is intended exclusively for aesthetic, experiential, and commercial decision-support applications, including user-driven evaluation and purchase decision-making with respect to artworks, fragrances, musical compositions, furniture, spatial design environments, apparel, footwear, headwear, jewelry, accessories, and related commercial aesthetic categories. In certain embodiments, the system is not configured to diagnose, treat, mitigate, prevent, or monitor any medical, psychological, or psychiatric condition and does not generate therapeutic, clinical, or behavioral interventions.

The structured comparative outputs generated by the system are configured to support user interpretation and, in certain embodiments, are not presented as ranked or prescriptive selections; instead, they present relational comparative representations of engagement distances that support user-driven evaluation and decision-making in aesthetic and experiential domains. In certain embodiments, the system is configured such that it does not generate system-directed ranked or prescriptive output selections on behalf of the user.

The structured comparative output supports the user's own interpretation and decision-making by presenting data encoding relative engagement distances in engagement-vector space. In certain embodiments, the system is configured such that the output generation pipeline does not apply, at any stage from engagement-vector comparison through structured comparative output generation, any model, parameter, or transformation derived from cross-user engagement data or trained on or derived from physiological responses, engagement data, or preference data from a plurality of users other than the individual user that directly or indirectly determines, contributes to, or modifies the structured comparative output for the individual user.

### C. Wearable Sensor Integration

The system integrates with one or more wearable sensing devices configured to acquire real-time time-series physiological signals from the individual user during interaction with physical, digital, or multisensory stimuli. Wearable sensing devices may include wrist-worn devices, finger-worn sensors, chest-mounted sensors, head-mounted devices, or research-grade physiological monitors.

Physiological signals acquired by the wearable sensing devices may include, without limitation, electrodermal activity, heart rate, heart-rate variability, skin temperature, photoplethysmography, respiration, accelerometry, motion data, or electroencephalography. Signals are acquired as time-series data streams across at least two distinct physiological signal modalities and may be time-aligned with identifiers corresponding to interaction sessions, stimulus exposures, spatial zones, or contextual metadata.

Physiological signal data may further be time-aligned, by at least one processor, with identifiers corresponding to artworks, products, materials, fragrances, soundscapes, spatial zones, or other stimulus-related metadata associated with the user interaction session. Such alignment supports subsequent association of computed engagement vectors with candidate stimuli for comparative output generation. In certain embodiments, when the stimulus comprises a fragrance, physiological signals are acquired during olfactory exposure to the fragrance product, including exposure to airborne volatile compounds during user interaction with the fragrance. The structured comparative output generated from such physiological signals does not produce a ranked fragrance recommendation list or a population-level olfactory preference model. In certain embodiments, the wearable sensing devices are configured only to acquire, buffer, or transmit physiological signal data. In such embodiments, execution of the trained computational inference model is performed exclusively on a computing device remote from the wearable sensing devices, including a mobile computing device, a server, a cloud-based computing system, or a combination thereof. The wearable devices do not execute the trained computational inference model locally. In certain embodiments, the wearable sensing devices may further receive and display structured comparative outputs generated by the remote computing device. Such display does not constitute execution of the trained computational inference model.

In one embodiment, the wearable sensing device comprises a wrist-worn form factor incorporating: an electrodermal activity sensor configured to acquire galvanic skin response signals from the palmar or volar wrist surface; a photoplethysmography sensor configured to acquire pulse waveform data and inter-beat interval measurements; and a skin temperature sensor configured to acquire peripheral skin temperature measurements. In certain embodiments, these sensors are co-integrated into a single wrist-worn device body configured to simultaneously acquire electrodermal activity, photoplethysmography, and skin temperature signals as time-series data streams during a single interaction session. In certain embodiments, at least two of the sensors are configured to acquire signals from overlapping or adjacent anatomical regions to support synchronized multimodal acquisition.

In another embodiment, the wearable sensing device comprises a chest-mounted sensor array incorporating an electrodermal activity sensor, a photoplethysmography sensor, and a respiration sensor co-integrated into a single wearable unit configured to simultaneously acquire electrodermal activity, photoplethysmography, and respiration signals during a single interaction session. In certain embodiments, the wearable sensing device further incorporates a motion sensor or accelerometer configured to generate motion data for motion artifact detection, motion artifact compensation, or as an additional physiological signal modality for multimodal feature extraction.

Wearable sensing devices suitable for use with the disclosed system include, without limitation, wrist-worn devices, finger-worn sensor rings, chest-mounted patches, ear-worn devices, and head-mounted devices incorporating electroencephalography sensors. These structural configurations are illustrative and non-limiting.

### D. Physiological Signal Preprocessing

Physiological signal data received from the wearable sensing devices are processed by a preprocessing module implemented by at least one processor. The preprocessing module applies modality-specific signal processing operations independently for each physiological signal modality to generate synchronized physiological signal segments.

Preprocessing operations may include noise filtering, motion artifact detection and compensation, baseline normalization, detrending, interpolation, temporal alignment, and temporal segmentation into fixed or adaptive time windows. In certain embodiments, adaptive baseline normalization is applied independently for each physiological signal modality to account for user-specific or session-specific physiological variability. Performing preprocessing operations independently for each modality prior to fusion preserves modality-specific signal characteristics and reduces cross-modal artifact contamination.

Temporal alignment is applied across the preprocessed signals from different modalities to generate synchronized physiological signal segments. The synchronized segments serve as input to the multimodal feature extraction module described in the corresponding section of this specification. Synchronization ensures that features extracted across modalities correspond to the same temporal intervals of user interaction with the stimulus.

The output of the preprocessing module comprises normalized, temporally aligned, and segmented physiological signal segments independently processed for each modality, suitable for multimodal feature extraction and computational inference.

### E. Multimodal Feature Extraction

A multimodal feature extraction module extracts features from the synchronized physiological signal segments across at least two distinct physiological signal modalities. Feature extraction operations may include computation of statistical descriptors, time-domain features, frequency-domain features, spectral features, or learned representations derived from the physiological signal data.

By way of example, electrodermal activity features may include tonic and phasic electrodermal activity components; heart-rate-variability features may include metrics such as RMSSD, SDNN, pNN50, low-frequency power, or high-frequency power; photoplethysmography features may include pulse amplitude, pulse waveform characteristics, and inter-beat intervals; and motion-related features may include activity level, movement patterns, or postural information. Electroencephalography features, when included as a modality, may include frequency-band power spectra, event-related components, or other signal-derived measures.

Feature extraction parameters may be independently learned or calibrated for each physiological signal modality during training of the computational inference model. Independent calibration of feature extraction parameters per modality accommodates differences in signal characteristics, measurement scale, noise profiles, and dynamic range across modalities. The extracted feature representations are assembled into a synchronized multimodal feature representation that serves as input to the inference engine.

### F. Inference Engine and Continuous Multi-Dimensional Engagement Vector

The inference engine is implemented by at least one processor and is configured to execute a trained computational inference model. The trained computational inference model performs non-linear multimodal fusion on the synchronized multimodal feature representation to generate a continuous multi-dimensional engagement vector representing a quantified physiological interaction response of the individual user to the stimulus. In certain embodiments, the engagement representation is described as a continuous multi-dimensional engagement vector; however, the engagement representation may more generally comprise any structured multi-dimensional representation encoding a quantified physiological interaction response, provided that such representation supports computation of similarity or distance measures in an engagement space.

The continuous multi-dimensional engagement vector is the primary output of the inference engine and constitutes a distinct data structure comprising multiple dimensions derived from the fused physiological signal features of at least two distinct physiological signal modalities. In certain embodiments, the engagement vector comprises a temporally evolving sequence of vectors representing changes in physiological interaction response over time during interaction with the stimulus. The engagement vector is structured to support downstream similarity or distance computation in engagement-vector space.

The trained computational inference model does not require an externally-determined stimulus efficacy label as a training input or reference. The model generates engagement vectors from physiological signal features directly, without applying separately ascertained stimulus effectiveness measures derived from independent behavioral testing, population polling, or cross-subject affective calibration.

The engagement vector does not constitute a classification of a discrete emotional or affective state. The inference engine does not apply a fixed-threshold mapping from physiological features to a predefined set of emotional categories, nor does it output discrete emotional labels. The engagement vector represents a continuous quantified physiological response, not a categorical affective assignment.

The engagement vector is not generated by comparison to stored baseline physiological responses associated with predefined emotional categories. The inference process does not require a database of emotional baseline signals against which user signals are matched to determine emotional state.

The engagement vector is not derived solely from neural similarity measures across subjects. The inference model generates the engagement vector from the multimodal physiological features of the individual user during interaction with the stimulus, not from the correlation or similarity of the user's neural signals to those of other subjects.

The trained computational inference model may comprise a neural network, including a deep learning model, a transformer, a recurrent neural network, a convolutional neural network, or a combination thereof. In certain embodiments, the model comprises an attention-based multimodal fusion operation that generates weighting over synchronized multimodal feature channels for continuous engagement-vector inference rather than classification of discrete emotional labels. The attention-based fusion operation does not produce a classification of discrete emotional or affective states. In certain embodiments, the trained computational model combines rule-based feature weighting with machine-learned parameters. In certain embodiments, the trained computational inference model is trained on a corpus of multimodal physiological signal data comprising time-series physiological signals from at least two distinct physiological signal modalities, acquired from one or more individual users during interaction with candidate stimuli across one or more interaction sessions. Training does not require externally labeled affective ground truth derived from population-level emotional annotation or cross-subject affective calibration. In certain embodiments, the model is trained using self-supervised, contrastive, reconstruction-based, or hybrid learning objectives applied to multimodal physiological signal segments, wherein the training objective operates over the multi-dimensional engagement-vector space rather than over discrete affective category labels. Feature extraction parameters and multimodal fusion weights are jointly optimized during training to minimize a loss function defined over the engagement-vector space. In certain embodiments, training data is accumulated across multiple individual user interaction sessions and used to iteratively refine the trained model through periodic retraining or fine-tuning cycles. In certain embodiments, the training corpus may comprise physiological signal data from a plurality of individual users; such multi-user training corpora are used exclusively to optimize inference model parameters and do not constitute cross-user aggregation of engagement outputs as described in the corresponding section of this specification. Trained model parameters are fixed after each training cycle and are not modified during runtime deployment. The inference model does not adapt its parameters based on individual user feedback, session outcomes, or engagement-vector outputs generated during runtime. User-specific adaptation is performed exclusively by a downstream personalization layer operating on inference-model-generated engagement vectors, as described in the corresponding section of this specification, and does not constitute modification of the trained computational inference model. In certain embodiments, the trained computational inference model parameters represent generalized feature transformation functions derived from training data and do not encode user-specific preference relationships, stimulus evaluation mappings, or individualized comparative output logic used in generation of structured comparative outputs for a specific individual user. In certain embodiments, the output of the inference engine is provided as input to optional downstream analysis layers separate from the inference engine itself. Such downstream layers may generate interpretive descriptors, visualization elements, or session summaries for presentation purposes. Such downstream outputs are not required outputs of the inference engine and do not form part of the primary engagement vector produced by the inference model.

The system does not modify, adapt, or control presentation of stimuli during an interaction session based on the engagement representation generated by the trained computational inference model.

### G. Reference Engagement Vector Library and Within-User Comparison

The system maintains a stored library of reference engagement vectors associated with the same individual user. Reference engagement vectors are generated by the trained computational inference model from physiological signals acquired during prior interaction sessions of the same individual user with candidate stimuli. Reference engagement vectors are stored in association with the candidate stimuli to which they correspond, enabling stimulus-specific within-user comparison. The stored reference engagement vectors are generated exclusively from physiological signals of the same individual user and are maintained without incorporation of engagement vectors, physiological measurements, or statistical parameters derived from any other user into the reference engagement vector library.

In the absence of prior stored reference engagement vectors for the same individual user —for example, during a user's first interaction session with the system —initialization reference vectors are generated from physiological signals acquired during the current interaction session. Initialization reference vectors serve as a baseline for comparison during the current session and may be retained as stored reference vectors for subsequent sessions. This first-session initialization mechanism ensures that the system is operable for new users who have not yet accumulated a stored library of reference engagement vectors. Such initialization reference vectors are generated exclusively from physiological signals of the same individual user and do not incorporate cross-user data.

The decision-support module computes one or more similarity or distance measures between the continuous multi-dimensional engagement vector generated for the current interaction and the stored reference engagement vectors associated with the same individual user. The comparison is performed within the engagement-vector space defined by the inference model output, without reference to population-level data, cross-subject norms, or externally derived stimulus efficacy parameters.

Similarity or distance measures may include, without limitation, cosine similarity, Euclidean distance, Mahalanobis distance, dynamic time warping, or other vector-space distance metrics applied to the continuous multi-dimensional engagement vectors. The choice of distance or similarity metric may be selected based on the structure and dimensionality of the engagement-vector space.

The within-user comparison architecture ensures that the computed similarity or distance measures reflect the individual user's own engagement patterns across candidate stimuli relative to that user's own prior engagement history. The system does not incorporate engagement vectors from other users into the computation of similarity or distance measures for the individual user's structured comparative output. The within-user comparison is performed without applying any model, parameter, or transformation derived from cross-user engagement data that determines or contributes to generation of the structured comparative output for the individual user.

### H. Structured Comparative Output

Based on the computed similarity or distance measures, the decision-support module generates a structured comparative output for the candidate stimuli. The structured comparative output comprises data encoding relative distances in a multi-dimensional engagement-vector space between one or more candidate stimuli and the individual user's reference engagement vectors. In certain embodiments, the structured comparative output is configured such that it does not generate an ordered or ranked list of candidate stimuli, and instead generates relational comparative representations encoding relative engagement distances in engagement-vector space. The system does not assign ranked positions, recommendation scores, or priority ordering to candidate stimuli. The structured comparative output presents engagement distance data in a relational format that supports the user's own interpretation and decision-making without prescribing a selection or preferred order. In certain embodiments, the structured comparative output is configured as a relational comparative representation encoding relative engagement distances in engagement-vector space, enabling comparison without assigning ranked positions, recommendation scores, or priority ordering. In certain embodiments, the structured comparative output comprises a graphical mapping of engagement vectors in a continuous multi-dimensional space, wherein the graphical mapping represents relative engagement distances between one or more candidate stimuli and the individual user's reference engagement vectors without assigning ranked positions, recommendation scores, or priority ordering. The graphical mapping may present engagement intensity, engagement consistency, engagement variation, or other engagement-response characteristics derived from the computed distance measures. In certain embodiments, the structured comparative output comprises a distance matrix encoding pairwise distances between engagement vectors associated with candidate stimuli.

The structured comparative output provides non-therapeutic comparative decision support. The output does not constitute a clinical, diagnostic, or therapeutic output, a population-level predictive output, or a system-generated product recommendation based on single-modality biometric mapping or population-level preference inference.

The structured comparative output is generated such that the output generation pipeline does not apply, at any stage from engagement-vector comparison through structured comparative output generation, any model, parameter, or transformation derived from cross-user engagement data or trained on or derived from physiological responses, engagement data, or preference data from a plurality of users other than the individual user that directly or indirectly determines, contributes to, or modifies the structured comparative output for the individual user, wherein this limitation does not preclude the use of multi-user data during model training or for aggregate analytics that do not generate individual user structured comparative outputs.

The structured comparative output is computed from candidate stimuli encompassing one or more individual stimuli. The system supports single-stimulus evaluation—in which the engagement vector for a single candidate stimulus is compared against the individual user's reference vectors—as well as multi-stimulus comparative evaluation—in which engagement vectors for multiple candidate stimuli are simultaneously compared against the individual user's reference vectors to generate a structured comparative engagement representation across those stimuli. In certain embodiments, only a single candidate stimulus is evaluated relative to the individual user's reference engagement representations. In certain embodiments, the physical stimulus is a commercial aesthetic object selected from, without limitation: an artwork, a fragrance, a musical composition, a furniture piece, a spatial design environment, a material sample, apparel, footwear, headwear, jewelry, or accessories. In such embodiments, the engagement-related response variables are generated through non-linear multimodal fusion of at least two peripheral physiological signal modalities. The structured comparative output evaluates candidate stimuli of the same category based on comparative engagement-response variables without generating a population-level classification model of the stimulus category. In certain embodiments, the structured comparative output comprises a distance matrix encoding pairwise distances between engagement vectors generated for each candidate stimulus within the interaction session, wherein each element of the distance matrix represents a computed similarity or distance measure between the engagement vectors of a pair of candidate stimuli in the multi-dimensional engagement-vector space, and wherein no element of the distance matrix constitutes a ranked position, recommendation score, or priority ordering. The distance matrix may be presented to the user as a relational data structure, a heatmap visualization, a spatial proximity map, or another non-ranked comparative representation. In certain embodiments, the structured comparative output may be presented via a user interface configured to display comparative relationships among multiple candidate stimuli simultaneously. In certain embodiments, the structured comparative output may be presented simultaneously or sequentially across candidate stimuli. In certain embodiments, the structured comparative output may exhibit observable characteristics including: (a) absence of ranked positions, recommendation scores, or priority ordering assigned to any candidate stimulus; (b) variation across different users interacting with the same candidate stimuli, reflecting individual user-specific physiological response patterns rather than population-level stimulus efficacy measures; and (c) dependence on within-user reference engagement vectors rather than cross-user aggregated data for generation of the comparative output. These characteristics arise from the within-user reference architecture and the absence of model, parameter, or transformation in the output generation pipeline.

### I. Personalization and Stimulus Conditioning

In certain embodiments, the system further comprises a personalization module configured to apply post-inference weighting, filtering, or clustering operations to historical engagement-related response variables associated with the individual user. The personalization module operates exclusively on stored engagement-related response variables output by the trained computational inference model for that user and does not incorporate cross-user data for generation of structured comparative outputs for the individual user. The personalization module operates as a post-inference processing layer separate from the trained computational inference model. The personalization module does not modify the parameters of the trained computational inference model during runtime; inference model parameters remain fixed across users, sessions, and deployments. In certain embodiments, generating the structured comparative output comprises filtering candidate stimuli using a user-specific preference profile derived from historical engagement-related response variables output by the inference engine for that user. The filtering applies user-specific weighting without crowd-based normalization, population-similarity matching, or cross-user collaborative filtering. The filtering operation does not modify parameters of the trained computational inference model. In certain embodiments, clustering is performed exclusively on inference-model-generated engagement vectors associated with a single user across multiple sessions to characterize intra-user engagement patterns. Clustering operates on engagement vectors generated by the trained computational inference model for that individual user and does not incorporate engagement vectors from other users into the clustering computation.

Stimulus-related conditioning may be applied by associating engagement-related response variables with contextual or stimulus metadata, including visual, auditory, olfactory, tactile, spatial, or environmental attributes. Such conditioning enables the generation of decision-support outputs that reflect both interaction response characteristics and stimulus attributes without altering the core inference process.

### J. Within-User Longitudinal Aggregation

In certain embodiments, continuous multi-dimensional engagement vectors generated by the trained computational inference model for the individual user are mathematically combined across multiple interaction sessions or stimulus exposures associated with the same individual user to generate aggregated engagement metrics. These aggregated metrics support non-therapeutic comparative output generation with respect to physical or aesthetic stimuli over longitudinal interaction histories.

The mathematical combination of within-user engagement vectors may comprise averaging, weighting, or other vector-based aggregation operations applied to the inference-model-generated engagement vectors. The aggregation is performed exclusively on inference-model-generated engagement vectors associated with the same individual user and is not performed on raw physiological measurements, single-modality biometric scores, or engagement vectors derived from a plurality of different users.

The within-user aggregation architecture generates longitudinal engagement representations that capture how a specific individual's engagement with a stimulus or class of stimuli evolves across multiple sessions, without reference to population-level engagement data or cross-user comparison.

### K. Cross-User Engagement Aggregation for Institutional Analytics

In certain embodiments, normalized continuous multi-dimensional engagement vectors generated by the trained computational inference model across a plurality of individual users are mathematically combined across multiple users, interaction sessions, or stimulus exposures to generate aggregated engagement metrics for non-therapeutic institutional analytics and comparative designation output generation with respect to physical or aesthetic stimuli. (See FIG. 9.) The aggregated engagement metrics are not used to generate individual user outputs and do not form a population-level stimulus efficacy model for predicting individual user responses. The mathematical combination may comprise averaging, weighting, or other vector-based aggregation operations applied to inference-model-generated engagement vectors. The aggregated output comprises comparative engagement metrics structured for institutional analysis of engagement patterns across users and is not used to modify stimuli, generate ranked recommendations, or produce clinical, diagnostic, or therapeutic outputs. The aggregated output does not constitute a population-level stimulus efficacy model. In certain embodiments, inference-model-generated engagement vectors are aggregated across multiple users or interaction sessions to support comparative evaluation, benchmarking, or designation of products, environments, or configurations.

Aggregation operations are performed on inference-model-generated engagement vectors, not on raw physiological signal data. Such aggregated outputs are generated for non-therapeutic institutional decision-support purposes and exclude individual-level identifying information.

Cross-user aggregation for institutional analytics is architecturally distinct from the within-user comparison described in the corresponding section of this specification. Within-user comparison generates individual user structured comparative outputs based on that user's own reference engagement vectors. Cross-user aggregation generates institutional-level engagement metrics by combining engagement vectors across a defined user population for purposes such as exhibition analytics, product benchmarking, or institutional readiness designation, without generating individual user outputs based on model, parameter, or transformation.

### L. Deployment Architectures

The system may be implemented using on-device, cloud-based, or hybrid computing architectures. In certain embodiments, processing steps are distributed across wearable sensing devices, mobile computing devices, and remote server systems, such that signal acquisition, preprocessing, feature extraction, inference, and structured comparative output generation are performed at different computing locations.

In one embodiment, the wearable sensing devices are configured only to acquire, buffer, or transmit physiological signal data, and execution of the trained computational inference model is performed exclusively on a computing device remote from the wearable sensing devices, including a mobile computing device, a server, a cloud-based computing system, or a combination thereof. In such embodiments, the inference engine, the decision-support module, and the structured comparative output generation pipeline execute on the remote computing device, not on the wearable sensing devices.

Distribution of processing steps does not alter the core inference pipeline or the functional operation of the system and may be selected based on latency, resource availability, privacy, or deployment considerations.

### M. Example Applications

The disclosed system may be deployed in a variety of physical, digital, or multisensory environments, including art galleries, museums, retail spaces, eCommerce or online product-evaluation platforms, fragrance laboratories, interior-design evaluation settings, material or product testing environments, virtual or augmented-reality systems, multisensory installations, or at-home product-sampling contexts. These embodiments are illustrative and non-limiting. In certain embodiments, comparative engagement outputs are generated across one or more candidate commercial aesthetic objects within a single interaction session of the same individual user, wherein the comparative output encodes relative differences in physiological interaction responses across the candidate commercial aesthetic objects. In certain embodiments, the system is deployed in fashion or luxury goods evaluation contexts, wherein candidate stimuli comprise apparel, footwear, headwear, jewelry, or accessories evaluated during in-situ trial interaction or at-home product-sampling sessions.

### N. Commercial Certification and Platform Embodiments

In certain commercial embodiments, the non-therapeutic decision-support outputs generated by the disclosed system may be utilized within branded or programmatic certification, readiness, or platform frameworks. Such frameworks are illustrative and non-limiting and are not required for operation of the invention.

In one illustrative embodiment, aggregated engagement-related response variables or designation outputs generated as described in the corresponding section of this specification may be used to support a certification program indicating that a product, environment, or multisensory experience satisfies predefined engagement or interaction criteria. Such a certification program may be referred to, for example, as Emotion Verified™, and may be associated with products or environments that meet comparative or benchmark non-therapeutic engagement thresholds derived from group-level aggregation.

In another illustrative embodiment, hardware-configuration or sensing-capability designation outputs may be used to indicate that a particular sensing configuration, device arrangement, or deployment environment is compatible with or suitable for operation of the disclosed system. Such a designation program may be referred to, for example, as Emotion Ready™, and may reflect readiness or compatibility based on non-therapeutic technical criteria rather than individual user affective states. In certain embodiments, cloud-based aggregation, inference, model-management, and application-programming-interface services or functionally equivalent distributed services described herein may be deployed as part of a centralized analytics and delivery platform. Such a platform may be referred to, for example, as Emotion Cloud™, and may provide access to aggregated engagement metrics, designation outputs, or decision-support services for multiple client devices or deployments.

The foregoing certification and platform embodiments are exemplary commercial implementations of the disclosed decision-support outputs and do not limit the scope of the invention. The claimed systems and methods are not dependent on any particular branding, certification program, or commercial designation framework.

1. A computer-implemented system for generating comparative non-therapeutic engagement outputs associated with an individual user's interaction with a physical, digital, or multisensory stimulus, the system comprising:
(a) one or more wearable sensing devices comprising physiological sensors configured to generate time-series physiological signals including at least two physiological signal modalities selected from electrodermal activity, heart rate, heart-rate variability, skin temperature, photoplethysmography, respiration, electroencephalography, or motion data;
(b) at least one processor configured to:
(i) receive the time-series physiological signals;
(ii) apply signal preprocessing operations comprising noise filtering, baseline normalization, temporal alignment, and temporal segmentation, the preprocessing being performed independently for each physiological signal modality, to generate synchronized physiological signal segments; and
(iii) extract a synchronized multimodal feature representation from the synchronized physiological signal segments, the multimodal feature representation comprising features derived from at least two distinct physiological signal modalities;
(c) an inference engine implemented by the at least one processor and configured to execute a trained computational inference model that performs non-linear multimodal fusion on the multimodal feature representation to generate a continuous multi-dimensional engagement vector representing a quantified physiological interaction response of the individual user to the stimulus, wherein the engagement vector does not constitute a classification of a discrete emotional or affective state and is not derived from comparison to predefined emotional category baselines; and
(d) a computer interface configured to output the continuous multi-dimensional engagement vector to a decision-support module configured to:
(i) compute one or more similarity or distance measures between the continuous multi-dimensional engagement vector and one or more stored reference engagement vectors associated with the individual user, wherein the stored reference engagement vectors are derived from physiological signals of the individual user obtained during prior interaction sessions or are initialized from physiological signals acquired during a current interaction session of the individual user and wherein the stored reference engagement vectors do not incorporate engagement vectors, physiological measurements, or statistical parameters derived from any other user; and
(ii) generate a structured comparative output for one or more candidate stimuli based on the computed similarity or distance measures, for presentation to the individual user, wherein the structured comparative output comprises data encoding relative distances in a multi-dimensional engagement-vector space and is configured as a relational comparative representation that enables comparison without assigning ranked positions, recommendation scores, or priority ordering;
wherein the structured comparative output is generated such that the output generation pipeline does not apply, at any stage from engagement-vector comparison through structured comparative output generation, any model, parameter, or transformation derived from cross-user engagement data or trained on or derived from physiological responses, engagement data, or preference data from a plurality of users other than the individual user that directly or indirectly determines, contributes to, or modifies the structured comparative output for the individual user;
wherein this limitation does not preclude the use of multi-user data during model training or for aggregate analytics that do not generate individual user structured comparative outputs; and
wherein the structured comparative output provides non-therapeutic comparative decision support and does not constitute a population-level predictive output and does not constitute a clinical, diagnostic, or therapeutic output.

2. A computer-implemented method for generating comparative non-therapeutic engagement outputs associated with an individual user's interaction with a physical, digital, or multisensory stimulus, the method comprising:
receiving, from one or more wearable sensing devices, time-series physiological signals including at least two physiological signal modalities;
applying signal preprocessing operations comprising noise filtering, baseline normalization, temporal alignment, and temporal segmentation independently for each physiological signal modality to generate synchronized physiological signal segments;
extracting a synchronized multimodal feature representation from the synchronized physiological signal segments, the multimodal feature representation comprising features derived from at least two distinct physiological signal modalities;
executing a trained computational inference model that performs non-linear multimodal fusion on the multimodal feature representation to generate a continuous multi-dimensional engagement vector representing a quantified physiological interaction response of the individual user to the stimulus, wherein the engagement vector does not constitute a classification of a discrete emotional or affective state and is not derived from comparison to predefined emotional category baselines;
computing one or more similarity or distance measures between the continuous multi-dimensional engagement vector and one or more stored reference engagement vectors associated with the individual user, wherein the stored reference engagement vectors are derived from physiological signals of the individual user obtained during prior interaction sessions or are initialized from physiological signals acquired during a current interaction session of the individual user and wherein the stored reference engagement vectors do not incorporate engagement vectors, physiological measurements, or statistical parameters derived from any other user;
generating a structured comparative output for one or more candidate stimuli based on the computed similarity or distance measures, for presentation to the individual user, wherein the structured comparative output comprises data encoding relative distances in a multi-dimensional engagement-vector space and is configured as a relational comparative representation that enables comparison without assigning ranked positions, recommendation scores, or priority ordering; wherein the structured comparative output is generated such that the output generation pipeline does not apply, at any stage from engagement-vector comparison through structured comparative output generation, any model, parameter, or transformation derived from cross-user engagement data or trained on or derived from physiological responses, engagement data, or preference data from a plurality of users other than the individual user that directly or indirectly determines, contributes to, or modifies the structured comparative output for the individual user, wherein this limitation does not preclude the use of multi-user data during model training or for aggregate analytics that do not generate individual user structured comparative outputs; and
wherein the structured comparative output provides non-therapeutic comparative decision support and does not constitute a population-level predictive output and does not constitute a clinical, diagnostic, or therapeutic output.

3. A non-transitory computer-readable medium storing instructions that, when executed by one or more processors, cause the one or more processors to perform operations for generating comparative non-therapeutic engagement outputs associated with an individual user's interaction with a physical, digital, or multisensory stimulus, the operations comprising:
receiving, from one or more wearable sensing devices, time-series physiological signals including at least two physiological signal modalities;
applying signal preprocessing operations comprising noise filtering, baseline normalization, temporal alignment, and temporal segmentation independently for each physiological signal modality to generate synchronized physiological signal segments;
extracting a synchronized multimodal feature representation from the synchronized physiological signal segments, the multimodal feature representation comprising features derived from at least two distinct physiological signal modalities;
executing a trained computational inference model that performs non-linear multimodal fusion on the multimodal feature representation to generate a continuous multi-dimensional engagement vector representing a quantified physiological interaction response of the individual user to the stimulus, wherein the engagement vector does not constitute a classification of a discrete emotional or affective state and is not derived from comparison to predefined emotional category baselines;
computing one or more similarity or distance measures between the continuous multi-dimensional engagement vector and one or more stored reference engagement vectors associated with the individual user, wherein the stored reference engagement vectors are derived from physiological signals of the individual user obtained during prior interaction sessions or are initialized from physiological signals acquired during a current interaction session of the individual user and wherein the stored reference engagement vectors do not incorporate engagement vectors, physiological measurements, or statistical parameters derived from any other user;
generating a structured comparative output for one or more candidate stimuli based on the computed similarity or distance measures, for presentation to the individual user, wherein the structured comparative output comprises data encoding relative distances in a multi-dimensional engagement-vector space and is configured as a relational comparative representation that enables comparison without assigning ranked positions, recommendation scores, or priority ordering; wherein the structured comparative output is generated such that the output generation pipeline does not apply, at any stage from engagement-vector comparison through structured comparative output generation, any model, parameter, or transformation derived from cross-user engagement data or trained on or derived from physiological responses, engagement data, or preference data from a plurality of users other than the individual user that directly or indirectly determines, contributes to, or modifies the structured comparative output for the individual user, wherein this limitation does not preclude the use of multi-user data during model training or for aggregate analytics that do not generate individual user structured comparative outputs; and wherein the structured comparative output provides non-therapeutic comparative decision support and does not constitute a population-level predictive output and does not constitute a clinical, diagnostic, or therapeutic output.

4. The system of claim 1, wherein the structured comparative output comprises a distance matrix encoding pairwise distances between engagement vectors associated with the one or more candidate stimuli, wherein each element of the distance matrix represents a computed similarity or distance measure between engagement vectors corresponding to different candidate stimuli, and wherein the distance matrix is configured to enable comparative evaluation without assigning ranked positions, recommendation scores, or priority ordering to the candidate stimuli.

5. The system of claim 1, wherein the structured comparative output is presented as a graphical spatial representation of engagement vectors in a multi-dimensional engagement-vector space, wherein the graphical spatial representation positions engagement vectors corresponding to different candidate stimuli according to relative distances between the engagement vectors, and wherein the graphical spatial representation enables visual comparison of the candidate stimuli without assigning ranked positions, recommendation scores, or priority ordering.

6. The system of claim 1, wherein the at least two physiological signal modalities comprise electrodermal activity and heart-rate variability.

7. The system of claim 1, wherein the stored reference engagement vectors associated with the individual user are initialized during an initial interaction session of the individual user with one or more stimuli.

8. The system of claim 1, wherein the structured comparative output is generated based on engagement vectors associated with a plurality of candidate stimuli evaluated within a single interaction session of the individual user.

9. The system of claim 1, wherein personalization of the structured comparative output is performed exclusively using engagement vectors associated with the individual user, without incorporation of engagement vectors, physiological measurements, or statistical parameters derived from any other user.

10. The system of claim 1, wherein execution of the trained computational inference model is performed on a computing device remote from the one or more wearable sensing devices, and wherein the one or more wearable sensing devices are configured to acquire, buffer, or transmit the physiological signals without performing inference processing.

11. The system of claim 1, wherein clustering is performed on engagement vectors associated with the individual user across multiple interaction sessions to identify intra-user engagement patterns, wherein the clustering does not generate cross-user preference groupings and does not incorporate engagement vectors, physiological measurements, or statistical parameters derived from any other user.

12. The system of claim 1, wherein engagement vectors associated with the individual user are aggregated across multiple interaction sessions to generate one or more aggregated engagement representations, wherein the aggregation is performed exclusively on engagement vectors associated with the individual user and does not incorporate engagement vectors, physiological measurements, or statistical parameters derived from any other user.

13. The system of claim 1, wherein feature extraction parameters are independently configured for each physiological signal modality to generate modality-specific features prior to multimodal fusion.

14. The system of claim 1, wherein the trained computational inference model performs non-linear multimodal fusion of features derived from the at least two physiological signal modalities to generate the engagement vector.

15. The system of claim 1, wherein the physical, digital, or multisensory stimulus comprises a commercial aesthetic object selected from the group consisting of an artwork, a fragrance, a musical composition, a furniture piece, a spatial design environment, a material sample, apparel, footwear, headwear, jewelry, or accessories.

16. A computer-implemented method for non-therapeutic commercial engagement evaluation, comprising:
receiving, from one or more wearable sensing devices worn by an individual user, physiological signals acquired during interaction of the individual user with one or more candidate commercial aesthetic objects;
generating, by at least one processor and based on the received physiological signals, a comparative engagement output encoding relative differences in physiological interaction responses of the individual user across the one or more candidate commercial aesthetic objects, wherein the comparative engagement output is generated based on a within-user comparison of physiological interaction responses of the same individual user acquired during interaction with the one or more candidate commercial aesthetic objects;
wherein the comparative engagement output is generated from engagement vectors derived from the physiological signals using a trained computational inference model, and wherein the engagement vectors do not constitute a classification of a discrete emotional or affective state and are not derived from comparison to predefined emotional category baselines;
providing the comparative engagement output to support a non-therapeutic purchase or selection decision of the individual user with respect to the one or more candidate commercial aesthetic objects;
wherein the comparative engagement output does not generate an ordered or ranked list of candidate commercial aesthetic objects, and does not generate a preference score, recommendation score, or priority ordering assigned to any candidate commercial aesthetic object;
wherein the comparative engagement output provides non-therapeutic comparative decision support and does not constitute a population-level predictive output and does not constitute a clinical, diagnostic, or therapeutic output; and
wherein the comparative engagement output is generated such that the output generation pipeline does not apply, at any stage from engagement-vector comparison through comparative engagement output generation, any model, parameter, or transformation derived from or trained on physiological responses, engagement data, or preference data from a plurality of users other than the individual user that directly or indirectly determines, contributes to, or modifies the comparative engagement output for the individual user.

17. The system of claim 1, wherein the structured comparative output is configured for presentation via a user interface, including simultaneous or sequential presentation of comparative information associated with the one or more candidate stimuli, without assigning ranked positions, recommendation scores, or priority ordering.

18. The system of claim 1, wherein the structured comparative output is configured such that it reflects individual user-specific physiological response patterns derived from engagement vectors associated with the individual user.

19. The system of claim 1, wherein the continuous multi-dimensional engagement vector encodes temporal dynamics of the physiological signals corresponding to the individual user's interaction with the stimulus.

20. The system of claim 1, wherein the structured comparative output evaluates candidate stimuli belonging to a same category of commercial aesthetic objects based on comparative engagement responses of the individual user.

21. The system of claim 1, wherein when the commercial aesthetic object is a fragrance, the physiological signals are acquired during olfactory exposure to the fragrance, and the structured comparative output does not generate a population-level olfactory preference model or a ranked fragrance recommendation list.

22. The system of claim 1, wherein the physiological signals are acquired exclusively via the one or more wearable sensing devices worn by the individual user.

23. The system of claim 1, wherein the system comprises:
(i) a trained computational inference model configured to generate engagement vectors from multimodal physiological signals, the trained computational inference model having parameters that remain fixed during runtime deployment; and
(ii) a post-inference personalization layer configured to operate exclusively on inference-model-generated engagement vectors to perform user-specific comparative evaluation without modifying parameters of the trained computational inference model.

24. The system of claim 1, wherein the similarity or distance measure comprises one or more of cosine similarity, Euclidean distance, Mahalanobis distance, or dynamic time warping applied to the continuous multi-dimensional engagement vectors.
